(19) United States
(12) Patent Application Publication
(10) Pub. No.: US 2025/0090168 A1
(43) Pub. Date: Mar. 20, 2025

(54) SMALL DIAMETER SURGICAL STAPLING DEVICE

(71) Applicant: Covidien LP, Mansfield, MA (US)

(72) Inventors: Zhaokai Wang, Shanghai (CN); Xiliang Zhang, Shanghai (CN); Jiangfeng Zhang, Shanghai (CN)

(73) Assignee: Covidien LP, Mansfield, MA (US)

(21) Appl. No.: 18/898,204

(22) Filed: Sep. 26, 2024

Related U.S. Application Data
(63) Continuation of application No. 16/857,923, filed on Apr. 24, 2020, now Pat. No. 12,133,650.
Application No. 16/857,923 is a continuation of application No. 15/576,291, filed on Nov. 22, 2017, now abandoned.

(51) Int. Cl. A61B 17/072; A61B 17/00; A61B 17/29

(52) U.S. Cl. CPC A61B 17/07207; A61B 2017/00473; A61B 2017/00526; A61B 2017/07214; A61B 2017/07257; A61B 2017/07271; A61B 2017/07278; A61B 2017/07285; A61B 2017/2937; A61B 2017/2944; A61B 2017/2947

(57) ABSTRACT

A surgical stapling device includes an outer tube having a proximal body portion and a distal channel portion. A distal end of the proximal body portion defines a pair of cutouts. An anvil assembly includes an anvil body defining a tissue contact surface and a pair of pivot members which are supported within the cutouts. A staple cartridge is supported in the distal channel portion of the outer tube and defines a tissue contact surface. The cutouts are dimensioned to allow movement of the pivot members within the cutouts to allow the tissue contact surface of the anvil body to move in relation to the tissue contact surface of the staple cartridge from a “parked position” in which the tissue contact surfaces are in juxtaposed engagement to a “clamped position” in which the tissue contact surfaces defining a tissue gap. A drive member is provided that is formed from sheet metal and includes upper and lower extending members that are positioned to engage the anvil body and the distal channel portion to define a maximum tissue gap between the tissue contact surfaces of the anvil body and the staple cartridge.

### CROSS-REFERENCE TO RELATED APPLICATION

This application is a continuation of U.S. patent application Ser. No. 16/857,923, filed Apr. 24, 2020, which is a continuation of U.S. patent application Ser. No. 15/576,291, filed Nov. 22, 2017, which is a National Stage of Application of PCT/CN2015/079659 filed May 25, 2015, and which each application is incorporated herein by reference in its entirety. To the extent a claim of priority is made to each of the above disclosed applications.

### BACKGROUND

### 1. Technical Field

The present disclosure relates to surgical stapling devices, and more particularly, to surgical stapling devices for laparoscopic or endoscopic use.

### 2. Background of Related Art

Surgical stapling devices for stapling tissue typically include a tool assembly having a staple cartridge, an anvil, and a knife that can be actuated to effect simultaneous dissection and suturing of tissue. When compared to traditional methods of manually applying threaded sutures to tissue, the use of surgical stapling devices to suture and dissect tissue has increased the speed of the suturing procedure and thus, minimized patient trauma.

In an endoscopic surgical procedure, a surgical stapler is inserted through a small incision in the skin or through a cannula to access a surgical site. Due to the complexity of known surgical stapling devices, a continuing need exists for small diameter surgical stapling devices that are suitable for insertion through a small diameter cannula, e.g., a 5 mm cannula, and can effectively suture and dissect tissue.

### SUMMARY

The present disclosure is directed to small diameter surgical stapling devices that can be inserted through a small diameter cannula. The stapling device includes a cartridge assembly and an anvil pivotally supported in relation to the cartridge assembly via a floating pivot member. This arrangement allows the anvil to be positioned in a “parked position” in which the anvil is juxtaposed engagement with the cartridge assembly. In the parked position, the dimension of the stapling device is minimized so that it can be inserted through a small diameter cannula.

More specifically, the present disclosure provides in one aspect a surgical stapling device including an outer tube, a tool assembly and a drive member. The outer tube has a proximal body portion and a distal channel portion. A distal end of the proximal body portion defines cutouts and the channel portion has an outer surface. The tool assembly has an anvil assembly including an anvil body defining a longitudinal axis and having an outer surface, a tissue contact surface and a pair of pivot members positioned proximally of the tissue contact surface. The pivot members are pivotally received within the cutouts of the proximal body portion of the outer tube. The cartridge assembly includes a staple cartridge and a plurality of staples. The staple cartridge defines a longitudinal axis, a tissue contact surface, and a plurality of staple retention pockets. Each of the staple retention pockets supports one of the plurality of staples. The staple cartridge is supported within the distal channel portion of the outer tube. The anvil body is pivotally supported on the outer tube in relation to the staple cartridge such that the tool assembly is movable between a closed position in which longitudinal axes of the anvil body and the staple cartridge are parallel and an open position in which the longitudinal axes of the anvil body and the staple cartridge define an acute angle. The drive member is supported within the staple cartridge and is translatable through the staple cartridge to eject the plurality of staples from the staple cartridge. The cutouts are dimensioned to allow movement of the pivot members within the cutouts such the when the tool assembly is in the closed position, the anvil body can be positioned in relation to the staple cartridge in a “parked position” in which the contact surfaces of the anvil body and the staple cartridge are in juxtaposed engagement to a “clamped position” in which the contact surfaces of the anvil body and the staple cartridge are spaced to define a tissue gap.

In some embodiments, the outer tube supports a coupling member that is adapted to releasably secure the surgical stapling device to an actuator for effecting movement of the drive member.

In certain embodiments, the drive member includes a distal working portion having upper and lower radially extending members. The upper radially extending member is positioned to engage the outer surface of the anvil body and the lower radially extending member is positioned to engage the outer surface of the distal channel portion of the outer tube.

In embodiments, the anvil body, staple cartridge and distal channel portion each define a longitudinal slot and the working portion of the drive member has an I-beam configuration including a vertical strut that extends through the slots of the anvil body, the staple cartridge, and the distal channel portion.

In some embodiments, the upper and lower radially extending members include an upper pair of radially extending members and a lower pair of radially extending members.

In certain embodiments, one of the pair of upper radially extending members extends from the vertical strut transversely in a first direction and the other of the pair of upper radially extending members extends from the vertical strut in a second direction opposite to the first direction. In addition, one of the pair of lower radially extending members extends from the vertical strut transversely in the first direction and the other of the pair of lower radially extending members extends from the vertical strut in the second direction opposite to the first direction.

In embodiments, the drive member is formed from sheet metal.

In some embodiments, the drive member supports a connector that is adapted to releasably couple the drive member to an actuator.

In certain embodiments, one of the pair of upper radially extending members is positioned distally of the other of the pair of the radially extending members and one of the pair of lower radially extending members is positioned distally of the other of the pair of the lower radially extending members.

In embodiments, the cartridge assembly further includes a plurality of pushers and a sled. Each of the plurality of staples is associated with a respective pusher and the sled is translatable through the cartridge into sequential engagement with the pushers in response to distal movement of the drive member to eject the plurality of staples from the staple cartridge.

In some embodiments, a biasing member is provided to urge the tool assembly toward the open position.

The present disclosure provides in another aspect a surgical stapling device including an outer tube, a tool assembly and a drive member. The outer tube has a proximal body portion and a distal channel portion having an outer surface. The tool assembly includes an anvil assembly and a cartridge assembly. The anvil assembly includes an anvil body defining a longitudinal axis. The anvil body has an outer surface, a tissue contact surface and a pair of pivot members positioned proximally of the tissue contact surface. The cartridge assembly includes a staple cartridge and a plurality of staples. The staple cartridge defines a longitudinal axis, a tissue contact surface and a plurality of staple retention pockets. Each of the staple retention pockets supports one of the plurality of staples. The staple cartridge is supported within the distal channel portion of the outer tube and the anvil body is pivotally supported on the outer tube in relation to the staple cartridge such that the tool assembly is movable between a closed position in which the longitudinal axes of the anvil body and the staple cartridge are parallel and an open position in which the longitudinal axes of the anvil body and the staple cartridge define an acute angle. The drive member is supported within the staple cartridge and is translatable through the staple cartridge to eject the plurality of staples from the staple cartridge. The drive member includes a body formed of sheet metal having a distal working portion including a pair of upper radially extending members and a pair of lower radially extending members. The pairs of upper and lower radially extending members are formed by bending upper and lower edges of the sheet metal.

In embodiments, a distal end of the proximal body portion defines cutouts and the pivot members are pivotally received within the cutouts.

In some embodiments, the pair of upper radially extending members is positioned to engage the outer surface of the anvil body and the pair of lower radially extending members is positioned to engage the outer surface of the channel portion of the outer tube to define a maximum tissue gap between the tissue contact surfaces of the anvil body and the staple cartridge when the anvil is in a closed position.

In certain embodiments, the anvil body, the staple cartridge and the distal channel portion each define a longitudinal slot and the working portion of the drive member has an I-beam configuration including a vertical strut that extends through the slots of the anvil body, staple cartridge, and the distal channel portion.

In some embodiments, one of the pair of upper radially extending members extends from the vertical strut transversely in a first direction and the other of the pair of upper radially extending members extends from the vertical strut in a second direction opposite to the first direction, and wherein one of the pair of lower radially extending members extends from the vertical strut transversely in the first direction and the other of the pair of lower radially extending members extends from the vertical strut in the second direction opposite to the first direction.

In certain embodiments, one of the pair of upper radially extending members is positioned distally of the other of the pair of the radially extending members and one of the pair of lower radially extending members is positioned distally of the other of the pair of lower radially extending members.

In embodiments, the cutouts are dimensioned to allow movement of the pivot members within the cutouts such that when the tool assembly is in the closed position, the anvil body can move in relation to the staple cartridge from a “parked position” in which the tissue contact surfaces of the anvil body and the staple cartridge are in juxtaposed engagement to a clamped position in which the tissue contact surfaces of the anvil body and the staple cartridge are spaced to define a tissue gap.

In embodiments, a biasing member is provided to urge the tool assembly toward the open position.

### BRIEF DESCRIPTION OF THE DRAWINGS

Various embodiments of the presently disclosed surgical stapling device are described herein with reference to the drawings, wherein:

FIG. 1 is a side, perspective view of one embodiment of the presently disclosed surgical stapling device in the form of a reload with the tool assembly in the open position;

FIG. 2 is an enlarged view of the indicated area of detail shown in FIG. 1;

FIG. 3 is a side perspective view of the reload shown in FIG. 1 secured to a powered handle assembly;

FIG. 4 is a side, perspective exploded view of the reload shown in FIG. 1;

FIG. 5 is a side, perspective, cutaway view of the proximal end of the anvil and a central portion of the outer tube of the reload shown in FIG. 1 with parts separated;

FIG. 6 is a side, perspective view of a central portion of the reload shown in FIG. 1;

FIG. 7 is a side perspective view of the central portion of the reload shown in FIG. 6 with the outer tube shown in phantom;

FIG. 8 is a side perspective view of the distal end of the knife bar including the I-beam and the sled with the parts separated;

FIG. 9 is a side view of the reload shown in FIG. 1 with the tool assembly in a parked position;

FIG. 10 is a side view of the reload shown in FIG. 9 as the reload is inserted into a 5 mm trocar;

FIG. 10A is a top view of the reload shown in FIG. 9;

FIG. 11 is an enlarged view of the indicated area of detail shown in FIG. 10;

FIG. 12 is a cross-sectional view taken along section line 12-12 of FIG. 10A;

FIG. 13 is a side view of the distal end of the reload shown in FIG. 1 with the tool assembly in the open position;

FIG. 14 is an enlarged view of the indicated area of detail shown in FIG. 13;

FIG. 15 is a side view of the distal end of the reload shown in FIG. 1 with the tool assembly in the clamped position;

FIG. 16 is an enlarged view of the indicated area of detail shown in FIG. 15;

FIG. 17 is a side cross-sectional view of the central portion of the reload showing the I-beam in the clamped position;

FIG. 18 is side, perspective view of the distal end of the reload shown in FIG. 1 with the tool assembly in the clamped position;

FIG. 19 is an enlarged view of the indicated area of detail shown in FIG. 18;

FIG. 20 is bottom, perspective view of the distal end of the reload shown in FIG. 1 with the tool assembly in the clamped position;

FIG. 21 is bottom, perspective view of the distal end of the reload shown in FIG. 1 with the tool assembly in the clamped position and the outer tube removed;

FIG. 22 is a side, cross-sectional view taken along section line 22-22 of FIG. 21; and

FIG. 23 is side, cross-sectional view of the tool assembly of the reload shown in FIG. 1 during a firing stroke of the reload.

### DETAILED DESCRIPTION OF EMBODIMENTS

Embodiments of the presently disclosed surgical stapling device will now be described in detail with reference to the drawings wherein like reference numerals designate identical or corresponding elements in each of the several views. In this description, the term “clinician” is used generally to refer to medical personnel including doctors, nurses, and support personnel. In addition, the term “endoscopic” procedures is used generally to refer to endoscopic, laparoscopic, arthroscopic, and any other surgical procedure performed through a small incision or a cannula inserted into a patient's body. Finally, the term “proximal” is used generally to refer to the portion of the apparatus that is closer to a clinician, while the term “distal” is used generally to refer to the portion of the apparatus that is farther from the clinician.

The present disclosure is directed to a small diameter surgical stapling device that can be inserted through a small diameter cannula, e.g., a 5 mm cannula. The stapling device includes a cartridge assembly and an anvil pivotally supported in relation to the cartridge assembly via a floating pivot member. The floating pivot member allows the anvil to be positioned in a “parked position” in which the anvil is juxtaposed engagement with the cartridge assembly as will be discussed in detail below. The surgical stapling device also includes a drive member of reduced size formed of sheet metal.

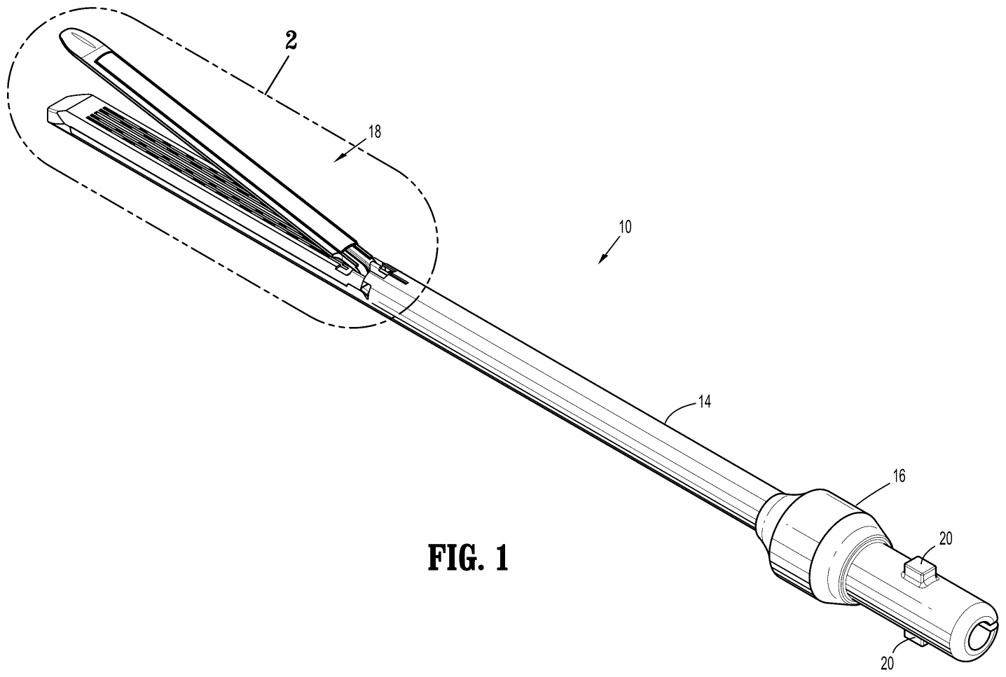

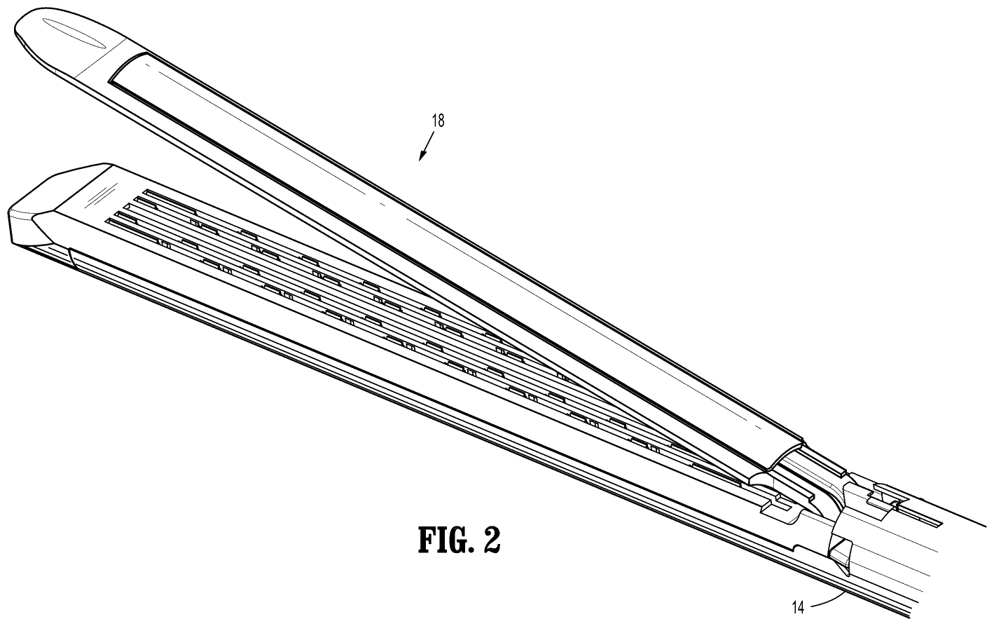

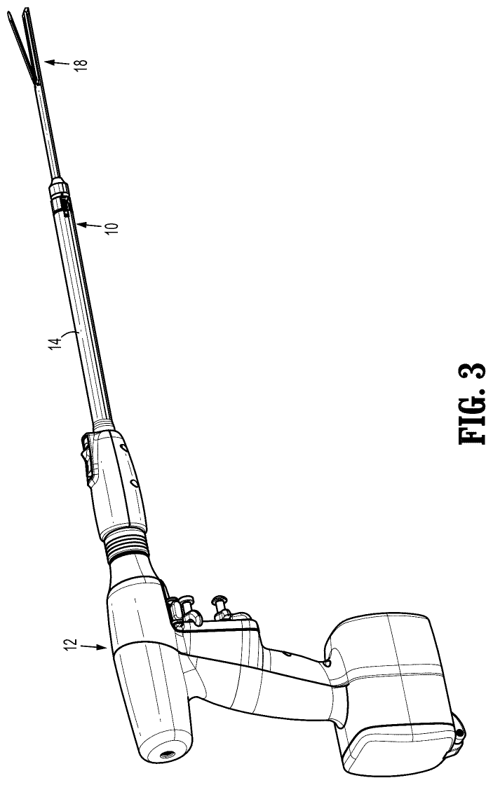

FIGS. 1-3 illustrate one embodiment of the presently disclosed surgical stapling device shown generally as 10. Surgical stapling device 10 is illustrated in the form of a reload that is adapted to be releasably coupled to an actuation device 12 (FIG. 3). Alternately, the reload 10 can be fixedly secured to the distal end of the actuation device 12. In embodiments, the actuation device 12 can be a powered actuation device as shown in FIG. 3. Alternately, the actuation device 12 can be a manually actuated actuation device, a robotically controlled actuation device, or any other suitable manually or electrically driven device configured to operate the stapling device 10.

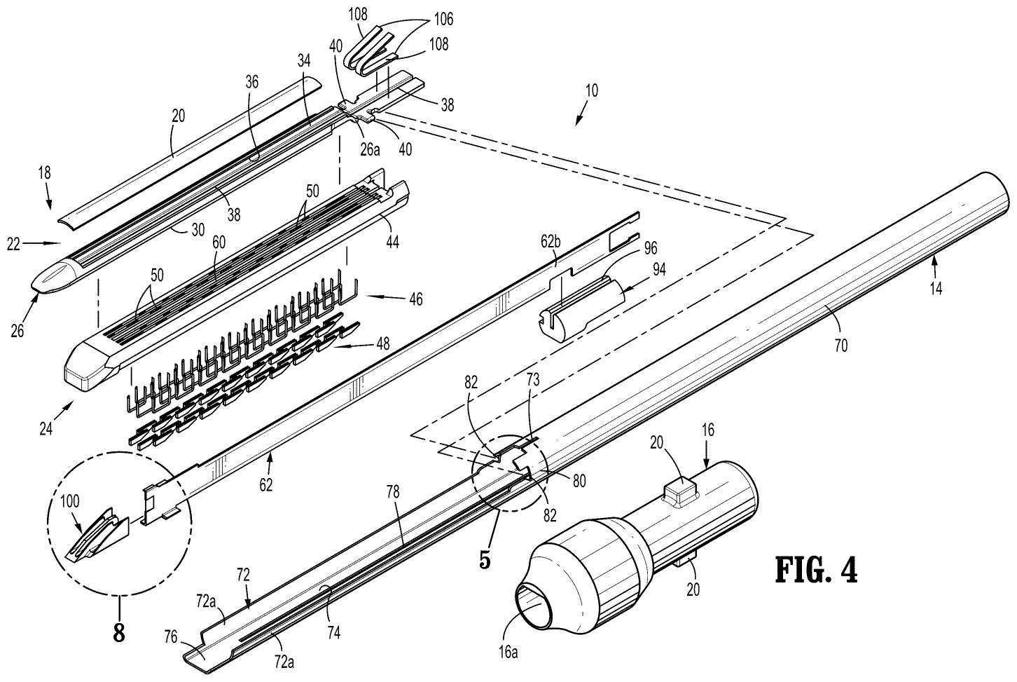

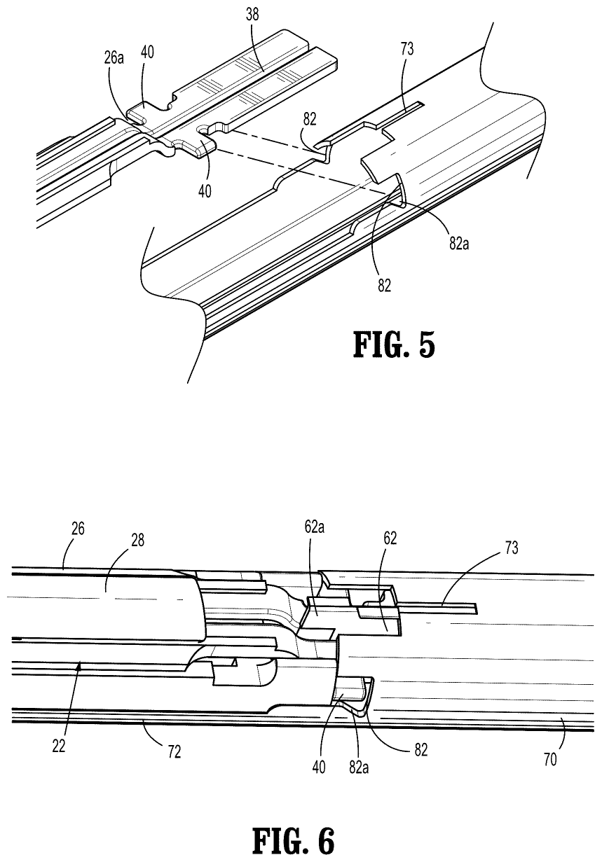

The reload 10 includes an outer tube 14, a coupling member 16 and a tool assembly 18. The coupling member 16 defines a longitudinal bore 16a (FIG. 4) that fixedly receives the proximal end of the outer tube 14 and includes a pair of diametrically opposed nubs 20. The nubs 20 are configured to engage the distal end of the actuation device 12 (FIG. 3) to secure the reload 10 to the actuation device 12 such as disclosed in U.S. Pat. No. 8,070,033 (“the '033 Patent”) which is incorporated herein by reference in its entirety. Alternately, the coupling member 16 can be integrally formed with the outer tube 14.

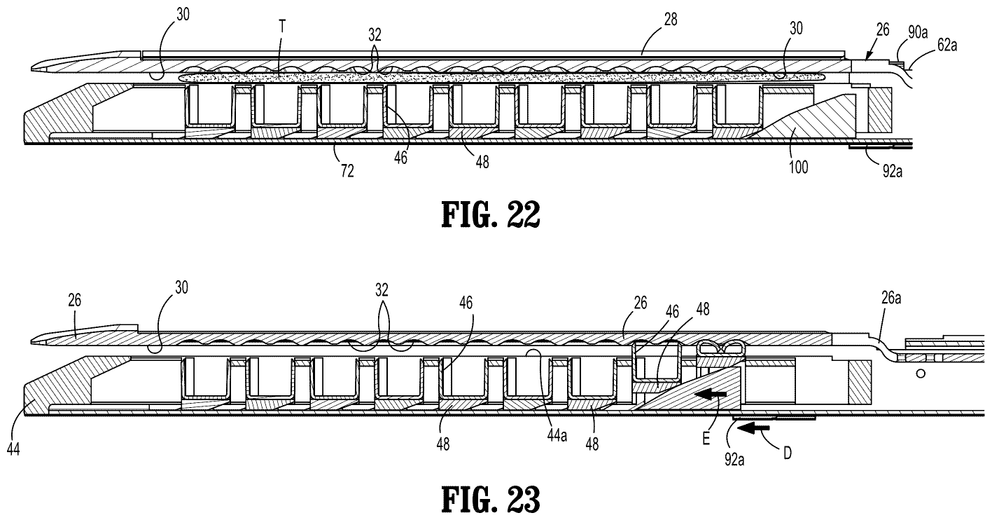

Referring to FIG. 4, the tool assembly 18 includes an anvil assembly 22 and a cartridge assembly 24. The anvil assembly 22 includes an anvil body 26 and a cover 28. The anvil body 26 includes an inner tissue contact surface 30 defining a plurality of staple deforming depressions 32 (FIG. 22), an outer cam surface 26a, and an outer surface 34 defining a longitudinal groove 36 that will be described in detail below. An elongated slot 38 extends through the anvil body 26 from a position adjacent a distal end of the anvil body 26 to the proximal end of the anvil body 26. The proximal end of the anvil body 26 includes a pair of pivot members 40 which will be described in detail below.

The cartridge assembly 24 includes a staple cartridge 44, a plurality of staples 46, and a plurality of staple pushers 48. The staple cartridge 44 defines a plurality of staple retention slots 50. Each staple retention slot 50 supports one of the plurality of staples 46 and is associated with one of the plurality of pushers 48 such that movement of the pusher 48 in relation to the retention slot 50 ejects a staple 46 from the retention slot 50. For a more detailed description of the interaction between the pushers 48 and the staples 46, see the '033 Patent. The cartridge 44 also defines a longitudinal slot 60 that receives the distal end of a drive member 62 as described in detail below.

Referring also to FIGS. 4 and 6, the outer tube 14 includes a proximal body portion 70 and a distal body portion 72. In embodiments, the outer tube 14 is substantially rigid and can be formed from a metal such as stainless steel. Alternately, the outer tube 14 can be formed of other substantially rigid materials suitable for medical use.

In embodiments, the proximal body portion 70 is substantially cylindrical and is dimensioned to slidably receive the drive member 62. A distal end 80 of the proximal body portion 70 defines upper and lower slots 73 which are described in detail below. The distal body portion 72 is substantially U-shaped and defines a channel portion 74 that receives the cartridge assembly 24. The distal body portion 72 includes sidewalls 72a that are positioned to support an outer wall of the staple cartridge 44. The staple cartridge 44 can be frictionally retained within the channel portion 74. Alternately, the staple cartridge 44 can be retained within the channel portion 74 using interlocking and/or snap-fit connections. A bottom wall 76 of the distal body portion 72 defines a longitudinal slot 78 that is aligned with the slot 38 in the anvil body 26 and with the slot 60 in the staple cartridge 44 such that the drive member 62 extends through each of the slots 38, 60, and 78 as described below.

The proximal body portion 70 has a distal end 80 that defines a pair of spaced cutouts 82 that are dimensioned to receive the pivot members 40 of the anvil body 26. The pivot members 40 are received within the respective cutouts 82 to facilitate pivotal movement of the anvil assembly 22 in relation to the cartridge assembly 24 and allow movement of the tool assembly from an “open position” in which longitudinal axes of the anvil and cartridge assemblies 22, 24 define an acute angle to a “closed position” in which the longitudinal axes of the anvil and cartridge assemblies 22, 24 are substantially parallel. In embodiments, the cutouts 82 are oversized in relation to the pivot members 40 such that pivot members 40 are able to move vertically within the cutouts 82. Vertical movement of the pivot members 40 within the cutouts 82 of the outer tube 14 allows a proximal end of the anvil body 26 to move in relation to the cartridge assembly 24 when the tool assembly 18 is in the “closed position” to allow the anvil body 26 to move between a “parked position” in juxtaposed engagement with the cartridge assembly 24 and a “clamped position” in which the anvil body 26 and the cartridge assembly 24 define a tissue gap.

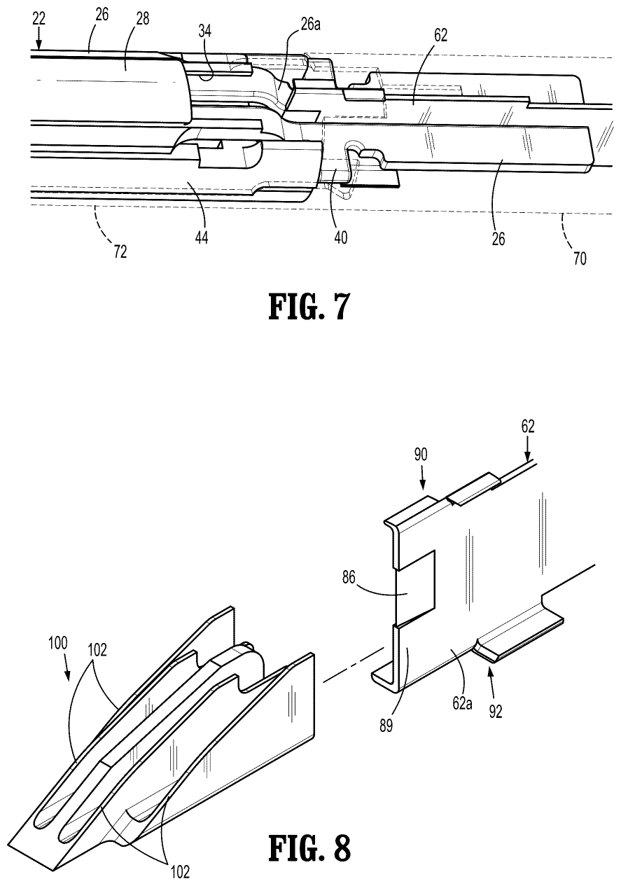

Referring to FIGS. 4, 7 and 8, the drive member 62 is elongated and has a reduced thickness to facilitate movement through the longitudinal slots 38, 60 and 78 of the anvil body 26, the staple cartridge 44, and the distal body portion 72, respectively. A distal end 62a of the drive member 62 supports a knife blade 86. In embodiments, the knife blade 86 is machined into the distal end of the drive member 62. Alternately, the knife blade 86 can be formed separately from the drive member 62 and secured to the drive member 62 such as by welding or the like. The drive member 62 includes a working distal end portion 62a having an I-beam configuration that includes vertical strut 89 and a pair of upper and lower extended members 90 and 92, respectively. The upper extended members 90 are positioned to slide along an outer surface of the anvil body 26 within the groove 36. Engagement of the upper extended members 90 with the cam surface 26a formed on the proximal end of the anvil body 26 causes the tool assembly 18 to move from the “open position” (FIG. 1) to the “clamped position” (FIG. 16). The cover 28 of the anvil assembly 22 encloses the upper extended members 90 within the groove 34. The lower extended members 92 are positioned to slide along an outer surface of the distal body portion 72 of the outer tube 14. In embodiments, the drive member 62 is formed from sheet metal and is deformed to form the upper and lower extended members 90 and 92 and machined to define the knife blade 86. More specifically, upper and lower ends of the sheet metal forming the distal end 62a of the drive member 62 can be bent outwardly to define the upper and lower extended members 90 and 92.

A proximal end 62b (FIG. 4) of the drive member 62 supports a connector 94. The connector 94 defines a longitudinal slot 96 that receives the drive member 62 to secure the drive member 62 to the connector 94. The connector 94 is configured to releasably engage a drive shaft (not shown) of the actuation device 12 (FIG. 3). For a more detailed discussion of a suitable connector 94, see the '033 Patent.

A sled 100 is supported within the staple cartridge 44 at a position distally of the distal end 62a of the drive member 62. The sled 100 includes cam members 102 that are configured to translate through channels (not shown) defined within the staple cartridge 44 into engagement with the pushers 48 to eject the staples 46 from the staple cartridge 44.

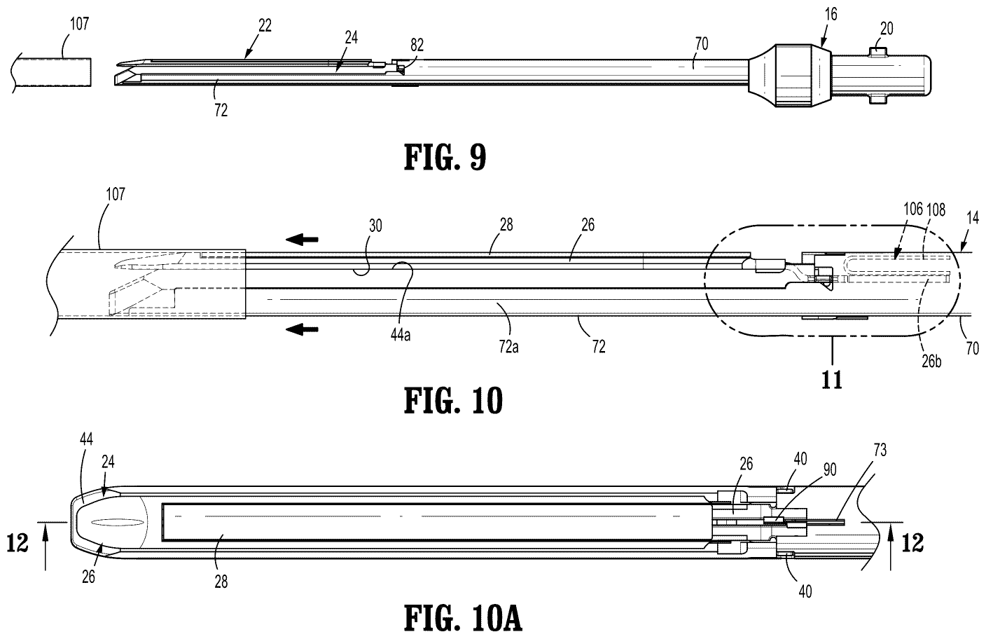

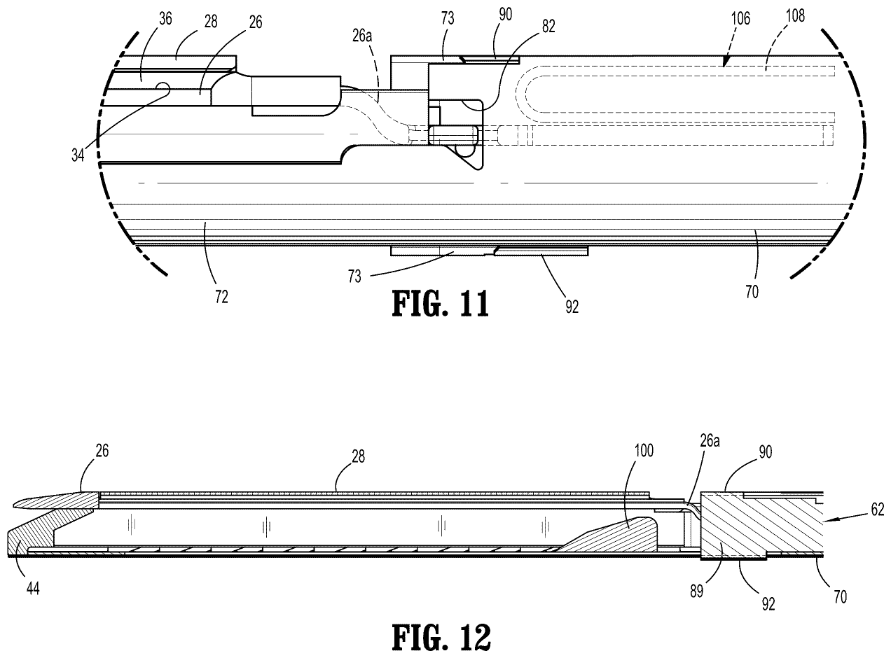

Referring to FIGS. 9-12, when the drive member 62 is in a fully retracted position, the vertical strut 89 of the drive member 62 (FIG. 8) extends through slots 73 defined in the distal end 80 of the proximal body portion 70 of the outer tube 14 such that the upper and lower extended members 90 and 92 are positioned along an outer surface of the outer tube 14 at a location spaced proximally of cam surface 26a of the anvil body 26. In this position, the anvil assembly 22 can be manually moved against the urging of a biasing member 106 to the “parked position” wherein the tissue contact surface 30 of the anvil body 26 is in juxtaposed engagement with the a tissue contact surface 44a of the staple cartridge 44 to minimize the outer diameter of the tool assembly 18. It is noted that surface to surface contact of the anvil body 26 and the staple cartridge 44 is possible because the pivot members 40 of the anvil body 26 float within the cutouts 82 formed in the distal face 80 of the proximal body portion 70 of the outer tube 14 to facilitate movement of the proximal end of the anvil body 26 to a position supported in a lower portion of the cutout 82 (FIG. 11). With the diameter of the tool assembly 18 minimized, the tool assembly 18 can be inserted through a small diameter cannula 107.

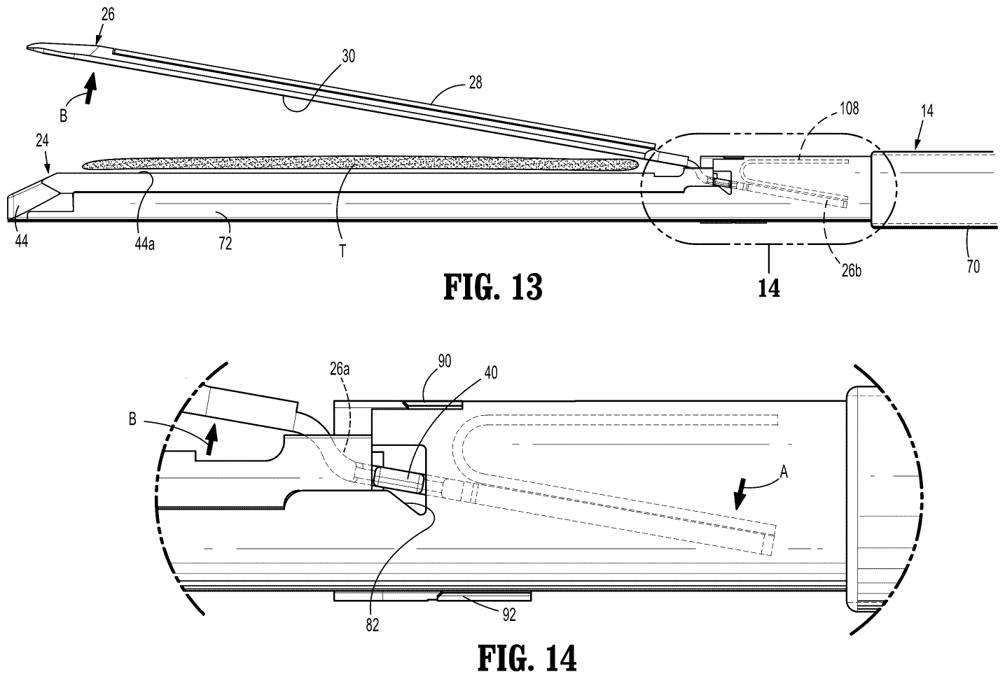

Referring to FIGS. 13 and 14, as discussed above, the tool assembly 18 includes a biasing member 106 including one or more springs 108. Each of the springs 108 is positioned between a proximal end 26b of the anvil body 26 and an inner surface of the outer tube 14 to urge the anvil body 26 towards the “open position”. The proximal end 26b of the anvil body 26 is positioned proximally of the pivot members 40 such that downward movement of the proximal end 26b of the anvil body 26 as indicated by the arrow A in FIG. 14 causes the tissue contact surface 30 of the anvil body 26 to move in a direction away from the tissue contact surface 44a of the staple cartridge 44 as indicated by the arrow “B” in FIG. 14.

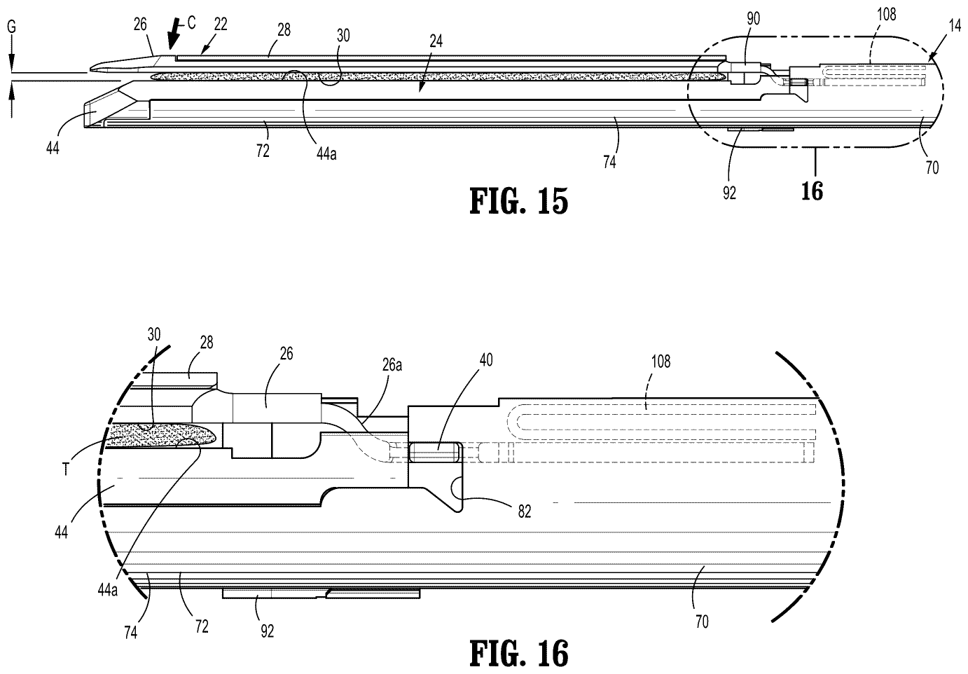

Referring to FIGS. 15-21, when the actuation device 12 (FIG. 3) is actuated to advance the drive member 62 within the cartridge assembly 24, the upper extended members 90 move distally along the outer surface of the anvil body 26 and pass over the cam surface 26a. Simultaneously, the lower extended members 92 translate along the outer surface of the channel portion 74 of the outer tube 14 to prevent outward deflection of the outer tube 14. Engagement of the upper extended members 90 with the cam surface 26a of the anvil body 26 causes the anvil assembly 22 to pivot to the “clamped position”. More specifically, when the upper extended members 90 (FIG. 19) of the drive member 62 engage the cam surface 26a of the anvil body 26, the pivot members 40 pivot within the cutouts 82 and the anvil assembly 22 moves in the direction indicated by arrow “C” in FIG. 15 to the “clamped position” about tissue “T”. As best seen in FIG. 16, as the tissue contact surface 30 of the anvil body 26 contacts tissue “T”, the proximal end of the anvil body 26 moves away from the staple cartridge 44 and the pivot members 40 move upwardly within the cutouts 82 of the outer tube 14 to allow the anvil assembly 22 to lift off of the staple cartridge 44 and move to the “clamped position” defining a tissue gap “G”. The tissue gap “G” accommodates placement of tissue between the tissue contact surfaces 30 and 44a of the anvil body 26 and the staple cartridge 44, respectively. A maximum tissue gap “G” is determined by the distance between the upper and lower radially extending members 90 and 92, respectively, which are positioned to move along the outer surfaces of the anvil assembly 22 and the channel portion 74 of the outer tube 14.

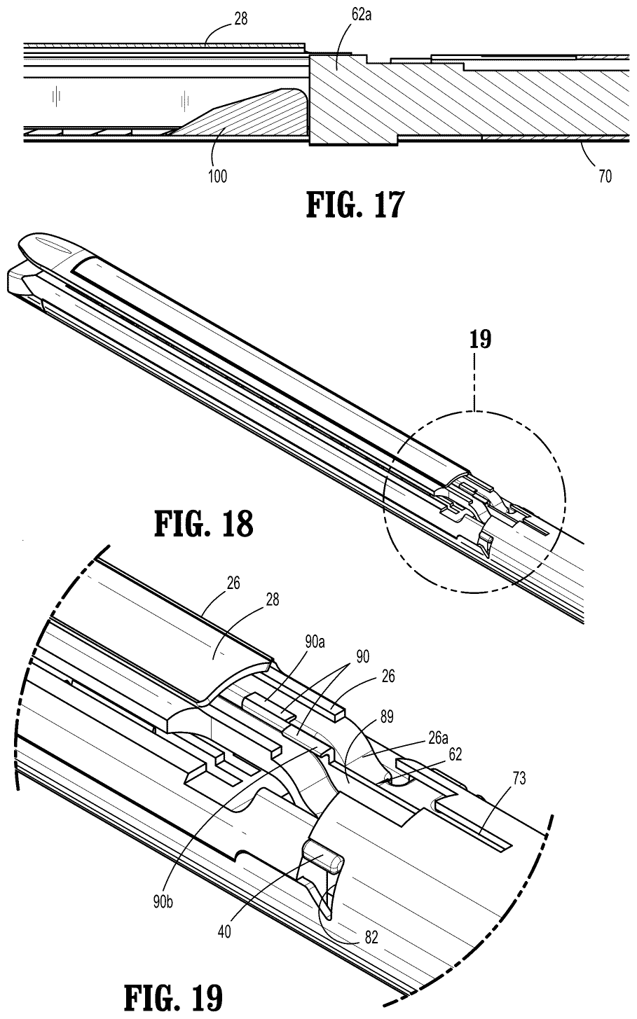

As illustrated in FIG. 17, the distal end 62a of the drive member 62 is positioned to engage the proximal end of the sled 100 such that distal movement of the drive member 62 causes corresponding distal movement of the sled 100 through the staple cartridge 44. When the tool assembly 18 is in the “clamped position”, the sled 100 is positioned proximally of the pushers 48 (FIG. 4).

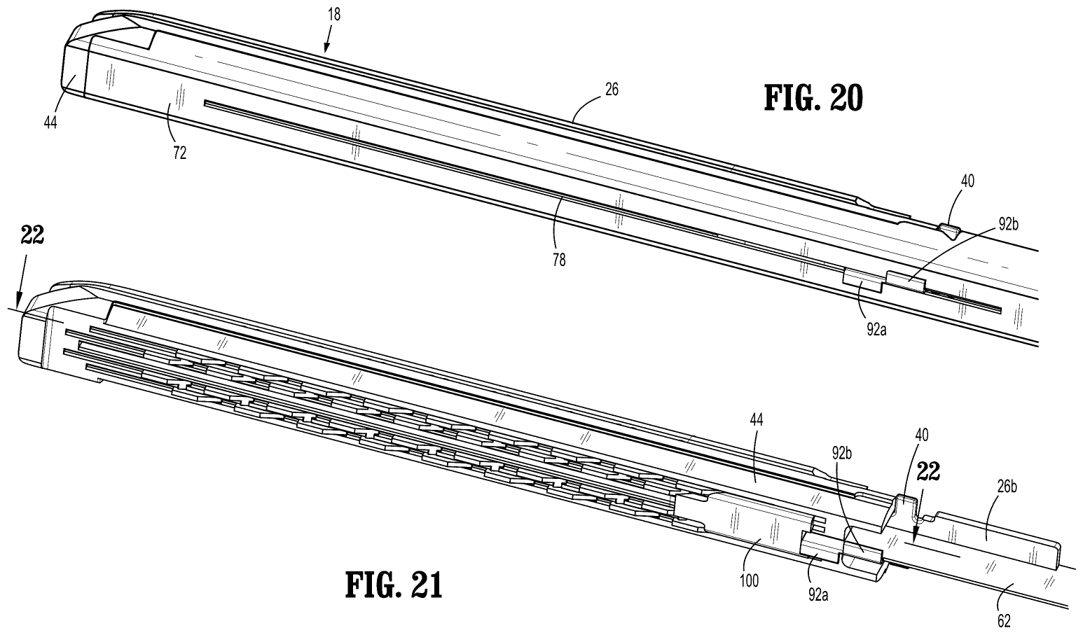

As illustrated in FIG. 19, the pair of upper extended members 90 include a first upper extended member 90a that extends transversely from the vertical strut 89 of the drive member 62 in a first direction along the outer surface of the anvil body 26 and a second upper extended member 90b that extends transversely from the vertical strut 89 in a second direction opposite to the first direction. The first upper extended member 92a is positioned distally of the second upper extended portion 90b. Similarly, as shown in FIGS. 20 and 21, the pair of lower extended members 92 include a first lower extended member 90a that extends transversely from the vertical strut 89 and the longitudinal slot 78 of the channel portion 74 in a first direction along the outer surface of the channel portion 74 of the outer tube 14 and a second lower extended member 92b that extends transversely from the vertical strut 89 and the longitudinal slot 78 in a second direction opposite to the first direction. The first lower extended portion 92a is positioned distally of the second lower extended member 92b. The pair of upper and lower extended members 90 and 92 are configured to provide secure engagement with the anvil assembly 22 and outer tube 14 to define the maximum tissue gap “G” (FIG. 15).

In embodiments, the upper and lower edges of the sheet metal forming the drive member 62 can be cut and bent in opposite directions to form the upper and lower pairs of extended members 90 and 92.

Referring to FIGS. 22 and 23, when the drive member 62 is advanced in the direction indicated by arrow “D” in FIG. 23 by the actuator 12 (FIG. 3), the distal working portion 62a of the drive member 62 advances the sled 100 in the direction indicated by arrow “E” through the staple cartridge 44 into sequential engagement with the pushers 48 to eject the staples 46 from the staple retention slots 50 of the staple cartridge 44. For a more detailed description of the operation of the sled 100 and the pushers 48, see the '033 Patent. As the working end portion 62a of the drive member 62 translates through the staple cartridge 44, the knife blade 86 (FIG. 4) on the distal end of the working end 62a of the drive member 62 dissects the tissue “T” positioned between the anvil assembly 22 and the staple cartridge 44.

In use, the tool assembly 18 of the surgical stapling device 10 is manually moved to the “parked position” and inserted into a small diameter cannula 107 (FIG. 10). As the tool assembly 18 of the surgical stapling device 10 exits the cannula 107 adjacent a surgical site, e.g., the abdomen, the tool assembly 18 will be moved to the “open position” by the biasing member 106, e.g., springs 108 (FIG. 13). In the “open position”, the tool assembly 18 is positioned to capture tissue to be treated “T”. Once the tool assembly 18 is properly positioned about tissue “T”, the actuation device 12 (FIG. 3) can be actuated to advance the drive member 62 to move the tool assembly 18 to the “clamped position” (FIG. 15). The tool assembly 18 can be inspected by the clinician to ensure that the tool assembly 18 is properly clamped about the tissue “T”. Thereafter, the actuation device 12 can be actuated to suture and dissect the tissue “T” (FIG. 23).

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another embodiment without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

1-19. (canceled)

20. A surgical stapling device, comprising:
an outer tube, comprising:
a proximal body portion having a cutout; and
a distal channel portion having an outer channel surface;
a tool assembly, comprising:
an anvil body pivotally coupled to the outer tube and defining a first longitudinal axis, the anvil body having an outer surface, a first tissue contact surface, and a pivot member received within the cutout; and
a staple cartridge disposed within the distal channel portion of the outer tube and defining a second longitudinal axis, the staple cartridge having a second tissue contact surface and a plurality of staple retention pockets configured to support a corresponding plurality of staples, with the anvil body being pivotable relative to the staple cartridge to define a closed position of the tool assembly, in which the first longitudinal axis is parallel to the second longitudinal axis, and to define an open position of the tool assembly, in which the first longitudinal axis defines an acute angle with the second longitudinal axis;
wherein, when the tool assembly is in the closed position, the anvil body is movable relative to the staple cartridge to define a parked position, in which the first tissue contact surface is juxtaposed with the second tissue contact surface and engaged therealong, and to define a clamped position, wherein the first tissue contact surface is in spaced alignment with the second tissue contact surface and defines a tissue gap therealong.

21. The surgical stapling device of claim 20, wherein the cutout is oversized in relation to the pivot member of the anvil body to define a floating pivot.

22. The surgical stapling device of claim 20, wherein the pivot member of the anvil body comprises a cam surface engaging the cutout such that the pivot member moves vertically when the anvil body moves between the parked position and the clamped position.

23. The surgical stapling device of claim 20, further including a biasing member engaged with the tool assembly and biasing the tool assembly toward the open position.

24. The surgical stapling device of claim 20, wherein the outer tube is of monolithic construction.

25. The surgical stapling device of claim 20, further comprising a drive member within the staple cartridge and translatable to eject the plurality of staples from the staple cartridge, the drive member comprising a body formed of sheet metal, the body having one or more extended members formed by bending an edge of the sheet metal.

26. The surgical stapling device of claim 25, wherein the one or more extended members comprises a pair of upper extended members positioned to engage the outer surface of the anvil body.

27. The surgical stapling device of claim 25, wherein the one or more extended members further comprises a pair of lower extended members positioned to engage the outer channel surface of the outer tube.

28. The surgical stapling device of claim 27, wherein the pair of upper extended members and the pair of lower extended members define a maximum tissue gap between the first tissue contact surface and the second tissue contact surface when the anvil assembly is in the clamped position.

29. A surgical stapling device comprising:
an outer tube, comprising:
a proximal body portion having a cutout; and
a distal channel portion having an outer channel surface; and
a tool assembly, comprising:
an anvil body pivotally coupled to the outer tube and having an outer surface, a first tissue contact surface, and a pivot member received within the cutout;
a staple cartridge disposed within the distal channel portion of the outer tube and having a second tissue contact surface and a plurality of staple retention pockets configured to support a corresponding plurality of staples, with the anvil body being pivotable relative to the staple cartridge to define a closed position and an open position for the tool assembly; and
a drive member supported within the staple cartridge and translatable to eject the plurality of staples from the staple cartridge, the drive member comprising a body formed of sheet metal, the body having one or more extended members formed by bending an edge of the sheet metal, the one or more extended members positioned to engage at least one of the anvil body or the outer tube to at least partially define a maximum tissue gap between the first tissue contact surface and the second tissue contact surface when the tool assembly is in the closed position.

30. The surgical stapling device of claim 29, wherein the one or more extended members comprises a pair of upper extended members positioned to engage the outer surface of the anvil body.

31. The surgical stapling device of claim 30, wherein the one or more extended members further comprises a pair of lower extended members positioned to engage the outer channel surface of the outer tube.

32. The surgical stapling device of claim 29, further including a biasing member engaged with the tool assembly and biasing the tool assembly toward the open position.

33. The surgical stapling device of claim 29, wherein the outer tube is of monolithic construction.

34. The surgical stapling device of claim 29, wherein the anvil body defines a first longitudinal axis and the staple cartridge defines a second longitudinal axis;
wherein, when the tool assembly is in the closed position, the first longitudinal axis is parallel to the second longitudinal axis.

35. The surgical stapling device of claim 34, wherein, when the tool assembly is in the closed position, the anvil body is movable relative to the staple cartridge to define:
a parked position, in which the first tissue contact surface is juxtaposed with the second tissue contact surface and engaged therealong; and
a clamped position, in which the first tissue contact surface is in spaced alignment with the second tissue contact surface and at least partially defines a tissue gap therealong.

36. The surgical stapling device of claim 35, wherein the tissue gap is the maximum tissue gap.

37. The surgical stapling device of claim 35, wherein the pivot member comprises a cam surface engaging the cutout such that the pivot member moves vertically when the anvil body moves between the parked position and the clamped position.

38. The surgical stapling device of claim 35, wherein the cutout is oversized in relation to the pivot member of the anvil body to define a floating pivot.

39. The surgical stapling device of claim 37, wherein, when the tool assembly is in the open position, the first longitudinal axis defines an acute angle with the second longitudinal axis.
